(19) United States
(12) Patent Application Publication
(10) Pub. No.: US 2021/0090691 A1
(43) Pub. Date: Mar. 25, 2021

(54) Cognitive System Candidate Response Ranking Based on Personal Medical Condition

(71) Applicant: International Business Machines Corporation, Armonk, NY (US)

(72) Inventors: Kristin E. McNeil, Charlotte, NC (US); Robert C. Sizemore, Fuquay-Varina, NC (US); David B. Werts, Charlotte, NC (US); Sterling R. Smith, Apex, NC (US)

(21) Appl. No.: 16/580,534

(22) Filed: Sep. 24, 2019

(51) Int. Cl. G16H 10/20; G16H 50/20; G16H 10/60; G06F 17/27

(52) U.S. Cl. CPC G16H 10/20; G06F 17/2735; G16H 10/60; G16H 50/20

(57) ABSTRACT

A mechanism is provided in a data processing system, wherein the at least one memory comprises instructions that are executed to implement a medical condition-based question answering (QA) system. The medical condition-based QA system processes a natural language input question about a patient to generate a set of candidate answers with an initial ranking of the candidate answers. A content indicator association component analyzes portions of content associated with each of the candidate answers in the set of candidate answers based on medical condition content indicator data structures corresponding to the one or more medical conditions associated with the patient to determine which portions of content match content indicators of the medical condition content indicator data structures. A response ranking component ranks candidate answers in the set of candidate answers based on the matching of content indicators of the medical condition content indicator data structures to the portions of content associated with the candidate answers to generate re-ranked candidate answers having a modified ranking. The medical condition-based QA system outputs the re-ranked candidate answers.

### BACKGROUND

The present application relates generally to an improved data processing apparatus and method and more specifically to mechanisms for cognitive system response ranking based on personal medical condition.

With the increased usage of computing networks, such as the Internet, humans are currently inundated and overwhelmed with the amount of information available to them from various structured and unstructured sources. However, information gaps abound as users try to piece together what they can find that they believe to be relevant during searches for information on various subjects. To assist with such searches, recent research has been directed to generating cognitive systems that may take an input question or request, analyze it, and return results indicative of the most probable response to the input question or request. Cognitive systems provide automated mechanisms for searching through large sets of sources of content, e.g., electronic documents, and analyze them with regard to an input question to determine an answer to the question and a confidence measure as to how accurate an answer is for answering the input question.

An electronic health record (EHR) or electronic medical record (EMR) is the systematized collection of patient and population electronically stored health information in a digital format. These records can be shared across different health care settings. Records are shared through network-connected, enterprise-wide information systems or other information networks and exchanges. EMRs may include a range of data, including demographics, social history, medical history, medication and allergies, immunization status, laboratory test results, radiology images, vital signs, personal statistics like age and weight, and billing information. A computerized healthcare cognitive system may be configured to assist in patient care based on EMR data for patients.

### SUMMARY

This Summary is provided to introduce a selection of concepts in a simplified form that are further described herein in the Detailed Description. This Summary is not intended to identify key factors or essential features of the claimed subject matter, nor is it intended to be used to limit the scope of the claimed subject matter.

In one illustrative embodiment, a method is provided in a data processing system comprising at least one processor and at least one memory. The at least one memory comprises instructions that are executed by the at least one processor to configure the at least one processor to implement a medical condition-based question answering (QA) system. The method comprises processing, by the medical condition-based QA system, a natural language input question about a patient to generate a set of candidate answers with an initial ranking of the candidate answers. The method further comprises analyzing, by a content indicator association component of the medical condition-based QA system, portions of content associated with each of the candidate answers in the set of candidate answers based on medical condition content indicator data structures corresponding to the one or more medical conditions associated with the patient to determine which portions of content match content indicators of the medical condition content indicator data structures. The method further comprises ranking, by a response ranking component of the medical condition-based QA system, candidate answers in the set of candidate answers based on the matching of content indicators of the medical condition content indicator data structures to the portions of content associated with the candidate answers to generate re-ranked candidate answers having a modified ranking. The method further comprises outputting, by the medical condition-based QA system, the re-ranked candidate answers.

In other illustrative embodiments, a computer program product comprising a computer useable or readable medium having a computer readable program is provided. The computer readable program, when executed on a computing device, causes the computing device to perform various ones of, and combinations of, the operations outlined above with regard to the method illustrative embodiment.

In yet another illustrative embodiment, a system/apparatus is provided. The system/apparatus may comprise one or more processors and a memory coupled to the one or more processors. The memory may comprise instructions which, when executed by the one or more processors, cause the one or more processors to perform various ones of, and combinations of, the operations outlined above with regard to the method illustrative embodiment.

These and other features and advantages of the present invention will be described in, or will become apparent to those of ordinary skill in the art in view of, the following detailed description of the example embodiments of the present invention.

### BRIEF DESCRIPTION OF THE DRAWINGS

The invention, as well as a preferred mode of use and further objectives and advantages thereof, will best be understood by reference to the following detailed description of illustrative embodiments when read in conjunction with the accompanying drawings, wherein:

FIG. 1 depicts a schematic diagram of one illustrative embodiment of a cognitive healthcare system in a computer network.

FIG. 2 is a block diagram of an example data processing system in which aspects of the illustrative embodiments are implemented;

FIG. 3 is an example diagram illustrating an interaction of elements of a healthcare cognitive system in accordance with one illustrative embodiment;

FIG. 4 illustrates a request processing pipeline for processing an input question in accordance with one illustrative embodiment;

FIG. 5 is a block diagram of a system for training a medical condition extraction model in accordance with an illustrative embodiment;

FIG. 6 is a block diagram of a cognitive computing system for response ranking based on personal medical condition in accordance with an illustrative embodiment;

FIG. 7 is a flowchart illustrating operation of a mechanism for training a medical condition extraction model in accordance with an illustrative embodiment; and

FIG. 8 is a flowchart illustrating operation of a mechanism for cognitive system candidate response ranking based on personal medical condition in accordance with an illustrative embodiment.

### DETAILED DESCRIPTION

Often users, such as patients or doctors, may submit questions to a cognitive computing system where the questions are directed to medical concepts. The cognitive computing system may return candidate answers or responses that are relevant to the input question, but the answers do not take into consideration the specific medical conditions of the patient as an additional factor for identifying answers that are of higher relevance to the particular user than others. As a result, users are provided with answers that may not be as relevant to the individual, and the users must then sift through candidate answers or otherwise reformulate input questions until they obtain pertinent answers.

There are cognitive computing systems that allow a user, such as a doctor to search patient electronic medical records (EMRs). However, such cognitive systems answer questions about the patient rather than answering more general questions taking the patient's medical condition into consideration. None of the known mechanisms specifically modify candidate answer result scoring based on the particular medical conditions associated with the user submitting the question, or a patient of the user in the case of a doctor being the user.

The illustrative embodiments provide a system that automatically learns a person's medical conditions, correlates that information with indicators of content that are specific to those medical conditions, and then uses those indicators to modify the ranking of candidate answer results generated by a cognitive computing system to an input question, so as to increase the ranking of candidate answers that have the content indicators correlated with the patient's specific medical conditions.

Before beginning the discussion of the various aspects of the illustrative embodiments in more detail, it should first be appreciated that throughout this description the term “mechanism” will be used to refer to elements of the present invention that perform various operations, functions, and the like. A “mechanism,” as the term is used herein, may be an implementation of the functions or aspects of the illustrative embodiments in the form of an apparatus, a procedure, or a computer program product. In the case of a procedure, the procedure is implemented by one or more devices, apparatus, computers, data processing systems, or the like. In the case of a computer program product, the logic represented by computer code or instructions embodied in or on the computer program product is executed by one or more hardware devices in order to implement the functionality or perform the operations associated with the specific “mechanism.” Thus, the mechanisms described herein may be implemented as specialized hardware, software executing on general purpose hardware, software instructions stored on a medium such that the instructions are readily executable by specialized or general-purpose hardware, a procedure or method for executing the functions, or a combination of any of the above.

The present description and claims may make use of the terms “a”, “at least one of”, and “one or more of” with regard to particular features and elements of the illustrative embodiments. It should be appreciated that these terms and phrases are intended to state that there is at least one of the particular features or elements present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

Moreover, it should be appreciated that the use of the term “engine,” if used herein with regard to describing embodiments and features of the invention, is not intended to be limiting of any particular implementation for accomplishing and/or performing the actions, steps, processes, etc., attributable to and/or performed by the engine. An engine may be, but is not limited to, software, hardware and/or firmware or any combination thereof that performs the specified functions including, but not limited to, any use of a general and/or specialized processor in combination with appropriate software loaded or stored in a machine-readable memory and executed by the processor. Further, any name associated with a particular engine is, unless otherwise specified, for purposes of convenience of reference and not intended to be limiting to a specific implementation. Additionally, any functionality attributed to an engine may be equally performed by multiple engines, incorporated into and/or combined with the functionality of another engine of the same or different type, or distributed across one or more engines of various configurations.

In addition, it should be appreciated that the following description uses a plurality of various examples for various elements of the illustrative embodiments to further illustrate example implementations of the illustrative embodiments and to aid in the understanding of the mechanisms of the illustrative embodiments. These examples are intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the examples provided herein without departing from the spirit and scope of the present invention.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

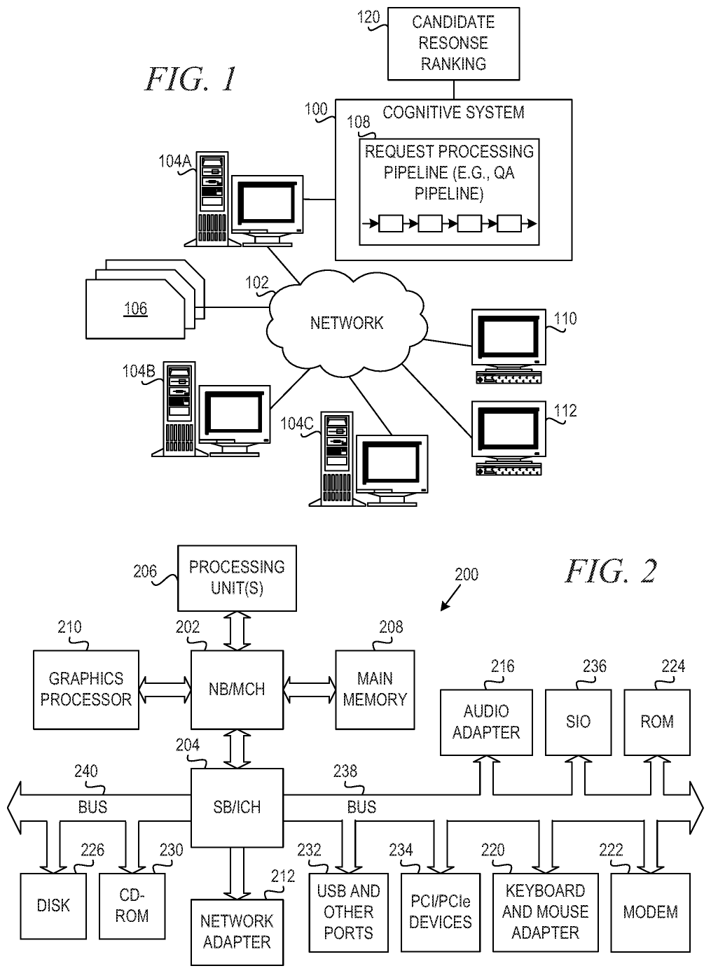

As noted above, the present invention provides mechanisms for graphical presentation of relevant information from electronic medical records. The illustrative embodiments may be utilized in many different types of data processing environments. In order to provide a context for the description of the specific elements and functionality of the illustrative embodiments, FIGS. 1-4 are provided hereafter as example environments in which aspects of the illustrative embodiments may be implemented. It should be appreciated that FIGS. 1-4 are only examples and are not intended to assert or imply any limitation with regard to the environments in which aspects or embodiments of the present invention may be implemented. Many modifications to the depicted environments may be made without departing from the spirit and scope of the present invention.

FIGS. 1-4 are directed to describing an example cognitive system for healthcare applications (also referred to herein as a “healthcare cognitive system”) which implements a request processing pipeline, such as a Question Answering (QA) pipeline (also referred to as a Question/Answer pipeline or Question and Answer pipeline) for example, request processing methodology, and request processing computer program product with which the mechanisms of the illustrative embodiments are implemented. These requests may be provided as structured or unstructured request messages, natural language questions, or any other suitable format for requesting an operation to be performed by the healthcare cognitive system. As described in more detail hereafter, the particular healthcare application that is implemented in the cognitive system of the present invention is a healthcare application for personalized patient engagement in care management using explainable behavioral phenotypes.

It should be appreciated that the healthcare cognitive system, while shown as having a single request processing pipeline in the examples hereafter, may in fact have multiple request processing pipelines. Each request processing pipeline may be separately trained and/or configured to process requests associated with different domains or be configured to perform the same or different analysis on input requests (or questions in implementations using a QA pipeline), depending on the desired implementation. For example, in some cases, a first request processing pipeline may be trained to operate on input requests directed to a first medical malady domain (e.g., various types of blood diseases) while another request processing pipeline may be trained to answer input requests in another medical malady domain (e.g., various types of cancers). In other cases, for example, the request processing pipelines may be configured to provide different types of cognitive functions or support different types of healthcare applications, such as one request processing pipeline being used for patient diagnosis, another request processing pipeline being configured for cognitive analysis of EMR data, another request processing pipeline being configured for patient monitoring, etc.

Moreover, each request processing pipeline may have its own associated corpus or corpora that it ingests and operates on, e.g., one corpus for blood disease domain documents and another corpus for cancer diagnostics domain related documents in the above examples. These corpora may include, but are not limited to, EMR data. The cognitive system may generate candidate answers to input questions and modify scoring of the candidate answers based on personal medical condition.

As will be discussed in greater detail hereafter, the illustrative embodiments may be integrated in, augment, and extend the functionality of these request processing pipeline mechanisms of a healthcare cognitive system with regard to candidate response ranking based on personal medical condition.

Thus, it is important to first have an understanding of how cognitive systems are implemented before describing how the mechanisms of the illustrative embodiments are integrated in and augment such cognitive systems and request processing pipeline mechanisms. It should be appreciated that the mechanisms described in FIGS. 1-4 are only examples and are not intended to state or imply any limitation with regard to the type of cognitive system mechanisms with which the illustrative embodiments are implemented. Many modifications to the example cognitive system shown in FIGS. 1-4 may be implemented in various embodiments of the present invention without departing from the spirit and scope of the present invention.

FIG. 1 depicts a schematic diagram of one illustrative embodiment of a cognitive system 100 implementing a request processing pipeline 108 in a computer network 102. The cognitive system 100 is implemented on one or more computing devices 104A-C (comprising one or more processors and one or more memories, and potentially any other computing device elements generally known in the art including buses, storage devices, communication interfaces, and the like) connected to the computer network 102. For purposes of illustration only, FIG. 1 depicts the cognitive system 100 being implemented on computing device 104A only, but as noted above the cognitive system 100 may be distributed across multiple computing devices, such as a plurality of computing devices 104A-C. The network 102 includes multiple computing devices 104A-C, which may operate as server computing devices, and 110-112 which may operate as client computing devices, in communication with each other and with other devices or components via one or more wired and/or wireless data communication links, where each communication link comprises one or more of wires, routers, switches, transmitters, receivers, or the like. In some illustrative embodiments, the cognitive system 100 and network 102 may provide cognitive operations including, but not limited to, request processing and cognitive response generation which may take many different forms depending upon the desired implementation, e.g., cognitive information retrieval, training/instruction of users, cognitive evaluation of data, or the like. Other embodiments of the cognitive system 100 may be used with components, systems, sub-systems, and/or devices other than those that are depicted herein.

The cognitive system 100 is configured to implement a request processing pipeline 108 that receive inputs from various sources. The requests may be posed in the form of a natural language question, natural language request for information, natural language request for the performance of a cognitive operation, or the like, and the answer may be returned in a natural language format maximized for efficient comprehension in a point-of-care clinical setting. For example, the cognitive system 100 receives input from the network 102, a corpus or corpora of electronic documents 106, cognitive system users, and/or other data and other possible sources of input. In one embodiment, some or all of the inputs to the cognitive system 100 are routed through the network 102. The various computing devices 104A-C on the network 102 include access points for content creators and cognitive system users. Some of the computing devices 104A-C include devices for a database storing the corpus or corpora of data 106 (which is shown as a separate entity in FIG. 1 for illustrative purposes only). Portions of the corpus or corpora of data 106 may also be provided on one or more other network attached storage devices, in one or more databases, or other computing devices not explicitly shown in FIG. 1. The network 102 includes local network connections and remote connections in various embodiments, such that the cognitive system 100 may operate in environments of any size, including local and global, e.g., the Internet.

In one embodiment, the content creator creates content in a document of the corpus or corpora of data 106 for use as part of a corpus of data with the cognitive system 100. The document includes any file, text, article, or source of data for use in the cognitive system 100. Cognitive system users access the cognitive system 100 via a network connection or an Internet connection to the network 102, and input requests to the cognitive system 100 that are processed based on the content in the corpus or corpora of data 106. In one embodiment, the requests are formed using natural language. The cognitive system 100 parses and interprets the request via a pipeline 108, and provides a response to the cognitive system user, e.g., cognitive system user 110, containing one or more answers to the question posed, response to the request, results of processing the request, or the like. In some embodiments, the cognitive system 100 provides a response to users in a ranked list of candidate responses while in other illustrative embodiments, the cognitive system 100 provides a single final response or a combination of a final response and ranked listing of other candidate responses.

The cognitive system 100 implements the pipeline 108 which comprises a plurality of stages for processing an input request based on information obtained from the corpus or corpora of data 106. The pipeline 108 generates responses for the input question or request based on the processing of the input request and the corpus or corpora of data 106.

In some illustrative embodiments, the cognitive system 100 may be the IBM Watson™ cognitive system available from International Business Machines Corporation of Armonk, N.Y., which is augmented with the mechanisms of the illustrative embodiments described hereafter. As outlined previously, a pipeline of the IBM Watson cognitive system receives an input question or request which it then parses to extract the major features of the question/request, which in turn are then used to formulate queries that are applied to the corpus or corpora of data 106. Based on the application of the queries to the corpus or corpora of data 106, a set of hypotheses, or candidate answers/responses to the input question/request, are generated by looking across the corpus or corpora of data 106 for portions of the corpus or corpora of data 106 (hereafter referred to simply as the corpus 106) that have some potential for containing a valuable response to the input question/response (hereafter assumed to be an input question). The pipeline 108 of the IBM Watson™ cognitive system then performs deep analysis on the language of the input question and the language used in each of the portions of the corpus 106 found during the application of the queries using a variety of reasoning algorithms.

The scores obtained from the various reasoning algorithms are then weighted against a statistical model that summarizes a level of confidence that the pipeline 108 of the IBM Watson cognitive system 100, in this example, has regarding the evidence that the potential candidate answer is inferred by the question. This process is to be repeated for each of the candidate responses to generate ranked listing of candidate responses, which may then be presented to the user that submitted the input request, e.g., a user of client computing device 110, or from which a final response is selected and presented to the user.

As noted above, while the input to the cognitive system 100 from a client device may be posed in the form of a natural language request, the illustrative embodiments are not limited to such. Rather, the input request may in fact be formatted or structured as any suitable type of request which may be parsed and analyzed using structured and/or unstructured input analysis, including but not limited to the natural language parsing and analysis mechanisms of a cognitive system to determine the basis upon which to perform cognitive analysis and providing a result of the cognitive analysis. In the case of a healthcare based cognitive system, this analysis may involve processing patient medical records, medical guidance documentation from one or more corpora, and the like, to provide a healthcare oriented cognitive system result.

In the context of the present invention, cognitive system 100 may provide a cognitive functionality for assisting with healthcare-based operations. For example, depending upon the particular implementation, the healthcare based operations may comprise patient diagnostics medical practice management systems, personal patient care plan generation and monitoring, patient electronic medical record (EMR) evaluation for various purposes, such as for identifying patients that are suitable for a medical trial or a particular type of medical treatment, or the like. Thus, the cognitive system 100 may be a healthcare cognitive system 100 that operates in the medical or healthcare type domains and which may process requests for such healthcare operations via the request processing pipeline 108 input as either structured or unstructured requests, natural language input questions, or the like.

As shown in FIG. 1, the cognitive system 100 is further augmented, in accordance with the mechanisms of the illustrative embodiments, to include logic implemented in specialized hardware, software executed on hardware, or any combination of specialized hardware and software executed on hardware, for implementing a candidate response ranking engine 120 for ranking candidate answers that have content indicators correlated with the user's specific medical conditions.

Candidate response ranking engine 120 improves performance of the cognitive computing system 100 by evaluating a person's medical condition and correlating that medical condition with content indicators indicating content that is most relevant to the medical conditions of the particular user. Cognitive computing system 100 answers input questions from a user, and candidate response ranking engine 120 re-ranks candidate answers generated by cognitive system 100 based on the particular medical conditions associated with the user or patient. In this way, the candidate answers corresponding to the content that is more relevant to the user's medical condition may have their ranking increased in the ranked listing of candidate answers. As a result, the more relevant answers to the specific medical conditions of the user are surfaced.

As noted above, the mechanisms of the illustrative embodiments are rooted in the computer technology arts and are implemented using logic present in such computing or data processing systems. These computing or data processing systems are specifically configured, either through hardware, software, or a combination of hardware and software, to implement the various operations described above. As such, FIG. 2 is provided as an example of one type of data processing system in which aspects of the present invention may be implemented. Many other types of data processing systems may be likewise configured to specifically implement the mechanisms of the illustrative embodiments.

FIG. 2 is a block diagram of an example data processing system in which aspects of the illustrative embodiments are implemented. Data processing system 200 is an example of a computer, such as server 104 or client 110 in FIG. 1, in which computer usable code or instructions implementing the processes for illustrative embodiments of the present invention are located. In one illustrative embodiment, FIG. 2 represents a server computing device, such as a server 104, which, which implements a cognitive system 100 and QA system pipeline 108 augmented to include the additional mechanisms of the illustrative embodiments described hereafter.

In the depicted example, data processing system 200 employs a hub architecture including North Bridge and Memory Controller Hub (NB/MCH) 202 and South Bridge and Input/Output (I/O) Controller Hub (SB/ICH) 204. Processing unit 206, main memory 208, and graphics processor 210 are connected to NB/MCH 202. Graphics processor 210 is connected to NB/MCH 202 through an accelerated graphics port (AGP).

In the depicted example, local area network (LAN) adapter 212 connects to SB/ICH 204. Audio adapter 216, keyboard and mouse adapter 220, modem 222, read only memory (ROM) 224, hard disk drive (HDD) 226, CD-ROM drive 230, universal serial bus (USB) ports and other communication ports 232, and PCI/PCIe devices 234 connect to SB/ICH 204 through bus 238 and bus 240. PCI/PCIe devices may include, for example, Ethernet adapters, add-in cards, and PC cards for notebook computers. PCI uses a card bus controller, while PCIe does not. ROM 224 may be, for example, a flash basic input/output system (BIOS).

HDD 226 and CD-ROM drive 230 connect to SB/ICH 204 through bus 240. HDD 226 and CD-ROM drive 230 may use, for example, an integrated drive electronics (IDE) or serial advanced technology attachment (SATA) interface. Super I/O (SIO) device 236 is connected to SB/ICH 204.

An operating system runs on processing unit 206. The operating system coordinates and provides control of various components within the data processing system 200 in FIG. 2. As a client, the operating system is a commercially available operating system such as Microsoft® Windows 10®. An object-oriented programming system, such as the Java™ programming system, may run in conjunction with the operating system and provides calls to the operating system from Java™ programs or applications executing on data processing system 200.

As a server, data processing system 200 may be, for example, an IBM® eServer™ System p® computer system, running the Advanced Interactive Executive (AIX®) operating system or the LINUX® operating system. Data processing system 200 may be a symmetric multiprocessor (SMP) system including a plurality of processors in processing unit 206. Alternatively, a single processor system may be employed.

Instructions for the operating system, the object-oriented programming system, and applications or programs are located on storage devices, such as HDD 226, and are loaded into main memory 208 for execution by processing unit 206. The processes for illustrative embodiments of the present invention are performed by processing unit 206 using computer usable program code, which is located in a memory such as, for example, main memory 208, ROM 224, or in one or more peripheral devices 226 and 230, for example.

A bus system, such as bus 238 or bus 240 as shown in FIG. 2, is comprised of one or more buses. Of course, the bus system may be implemented using any type of communication fabric or architecture that provides for a transfer of data between different components or devices attached to the fabric or architecture. A communication unit, such as modem 222 or network adapter 212 of FIG. 2, includes one or more devices used to transmit and receive data. A memory may be, for example, main memory 208, ROM 224, or a cache such as found in NB/MCH 202 in FIG. 2.

Those of ordinary skill in the art will appreciate that the hardware depicted in FIG. 2 may vary depending on the implementation. Other internal hardware or peripheral devices, such as flash memory, equivalent non-volatile memory, or optical disk drives and the like, may be used in addition to or in place of the hardware depicted in FIG. 2. Also, the processes of the illustrative embodiments may be applied to a multiprocessor data processing system, other than the SMP system mentioned previously, without departing from the spirit and scope of the present invention.

Moreover, the data processing system 200 may take the form of any of a number of different data processing systems including client computing devices, server computing devices, a tablet computer, laptop computer, telephone or other communication device, a personal digital assistant (PDA), or the like. In some illustrative examples, data processing system 200 may be a portable computing device that is configured with flash memory to provide non-volatile memory for storing operating system files and/or user-generated data, for example. Essentially, data processing system 200 may be any known or later developed data processing system without architectural limitation.

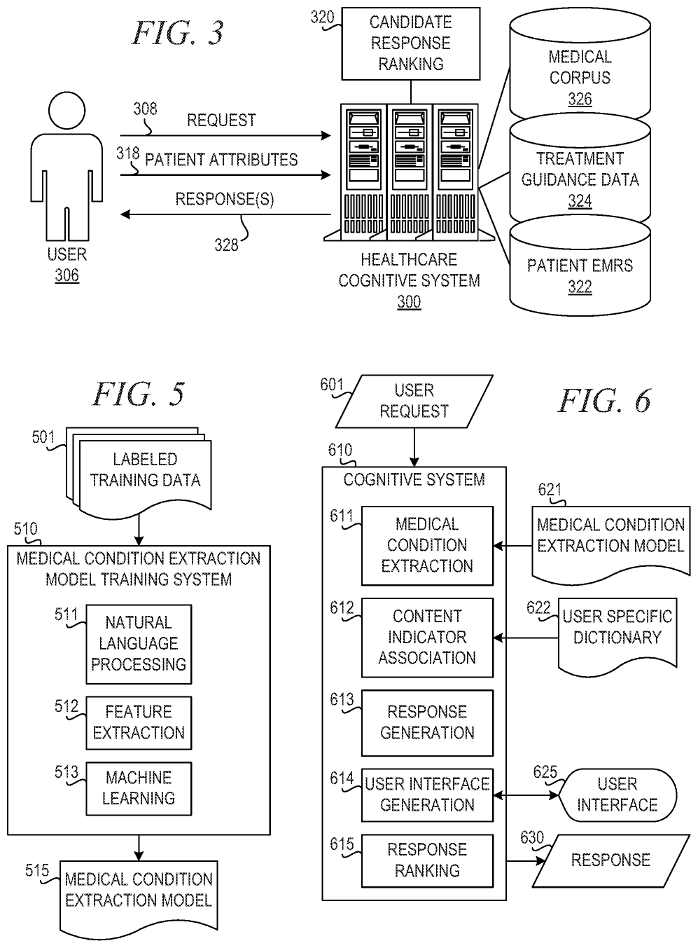

FIG. 3 is an example diagram illustrating an interaction of elements of a healthcare cognitive system in accordance with one illustrative embodiment. The example diagram of FIG. 3 depicts an implementation of a healthcare cognitive system 300 that is configured to provide candidate responses to questions or requests based on a patient's particular medical conditions. However, it should be appreciated that this is only an example implementation and other healthcare operations may be implemented in other embodiments of the healthcare cognitive system 300 without departing from the spirit and scope of the present invention.

Moreover, it should be appreciated that while FIG. 3 depicts the user 306 as a human figure, the interactions with user 306 may be performed using computing devices, medical equipment, and/or the like, such that user 306 may in fact be a computing device, e.g., a client computing device. For example, interactions between the user 306 and the healthcare cognitive system 300 will be electronic via a user computing device (not shown), such as a client computing device 110 or 112 in FIG. 1, communicating with the healthcare cognitive system 300 via one or more data communication links and potentially one or more data networks.

As shown in FIG. 3, in accordance with one illustrative embodiment, the user 306 submits a request 308 to the healthcare cognitive system 300, such as via a user interface on a client computing device that is configured to allow users to submit requests to the healthcare cognitive system 300 in a format that the healthcare cognitive system 300 can parse and process. The request 308 may include, or be accompanied with, information identifying patient attributes 318. These patient attributes 318 may include, for example, an identifier of the patient, social history, and demographic information about the patient, symptoms, and other pertinent information obtained from responses to questions or information obtained from medical equipment used to monitor or gather data about the condition of the patient. Any information about the patient that may be relevant to a cognitive evaluation of the patient by the healthcare cognitive system 300 may be included in the request 308 and/or patient attributes 318.

The healthcare cognitive system 300 provides a cognitive system that is specifically configured to perform an implementation specific healthcare oriented cognitive operation. In the depicted example, this healthcare oriented cognitive operation is directed to assist the user 306 in providing candidate answers to input questions ranked based on the patients' particular medical conditions. The healthcare cognitive system 300 operates on the request 308 and patient attributes 318 utilizing information gathered from the medical corpus and other source data 326, treatment guidance data 324, and the patient EMRs 322 associated with the patient to generate responses 328. The responses 328 may be presented in a ranked ordering with associated supporting evidence, obtained from the patient attributes 318 and data sources 322-326, indicating the reasoning as to why the response is being provided.

Note that EMR data 322 or data presented to the user may come from home readings or measurements that the patient makes available and are collected into EMR data 322.

In accordance with the illustrative embodiments herein, the healthcare cognitive system 300 is augmented to include a candidate response ranking engine 320 for modifying scoring of candidate responses based on the patient's medical conditions. Candidate response ranking engine 320 utilizes a cognitive computing system 300 evaluation of a patient's electronic medical records 322, social networking interactions, electronic mail communications, instant messaging communications, and the like, to determine the medical conditions associated with a particular user to generate a listing of one or more medical conditions. These medical conditions may be any condition that affects the health of the user, including medical problems (e.g., obesity, diabetes, heart conditions, high blood pressure, etc.), behavior conditions (e.g., negative habits, overeating, alcoholism, drug addiction, etc.), and psychological conditions (e.g., phobias, compulsions, etc.). The cognitive computing system 300 may process the patient's electronic medical records 322 using natural language processing, identification of recognizable medical codes, and the like, to identify these medical conditions that are associated with the patient or user. Moreover, the medical conditions may be specific sub-types, e.g., particular type of cancer, particular cancer stage, particular type of diabetes, particular symptoms experienced by the patient, etc.

Candidate response ranking engine 320 correlates the medical conditions associated with the user with medical conditions for which data structures have been defined that specify the particular terms/phrases, metadata, or other indicators of content that are indicative of content of particular interest to users having the corresponding medical conditions. Content in a corpus or content utilized by a cognitive computing system for answering input questions from users may be analyzed based on these content indicators to identify those portions of content that are more relevant than others to the user's specific medical conditions and modify the ranking of candidate answers to input questions that arise from such content accordingly.

Candidate response ranking engine 320 may generate a user specific dictionary data structure specifying the particular content indicators for the specific user based on the correlation of the medical conditions of the user with the predefined data structures. The user specific dictionary data structure may be installed in, or is otherwise accessible to, a cognitive computing system, such as healthcare cognitive system 300, which may then re-rank candidate answer results based on a correlation of the user specific dictionary data structure with content from which the candidate answers were generated, or which serve as evidence to support a scoring of the candidate answers, e.g., by matching of terms/phrases in the user specific dictionary data structure with terms/phrases in the content corresponding to the candidate answers. The degree of matching and/or number of instances of matching may be used as an additional mechanism for modifying the scores or rankings of candidate answers to thereby modify the original score or ranking based on the correlation of candidate answers with the medical conditions of the user. In some embodiments, such scoring or re-ranking may be performed specifically in response to an analysis of the original input question to determine whether the search input question is directed to a medical domain or a domain corresponding to the patient's medical conditions.

Thus, for example, a cancer patient may input a natural language question directed to cancer treatment trials, and the mechanisms of the illustrative embodiments may rank candidate answers based on the particular patient's type of cancer, cancer stage, symptoms, or the like, and the appearance of corresponding terms/phrases in content from which the candidate answers were generated or which were used as supportive evidence for the scoring of the candidate answers.

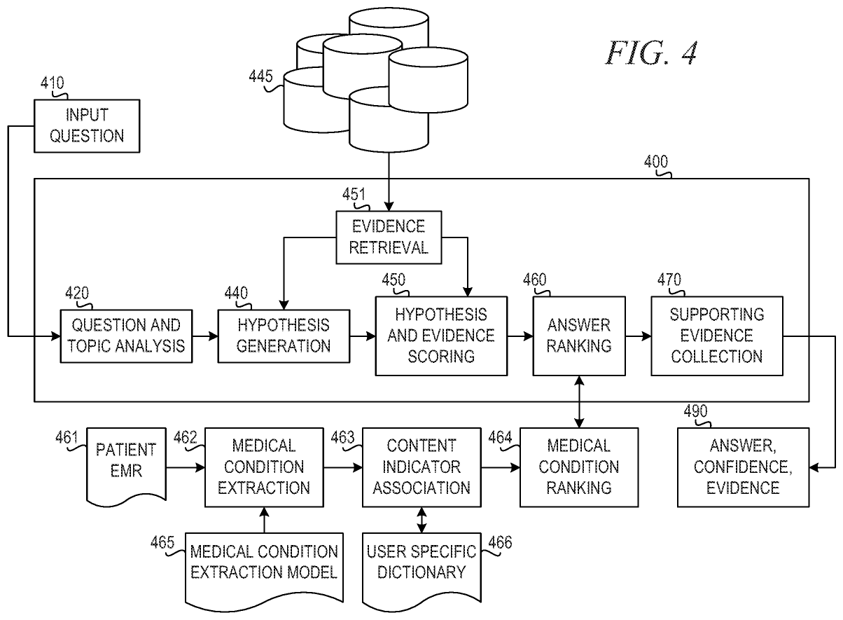

FIG. 4 illustrates a request processing pipeline for processing an input question in accordance with one illustrative embodiment. The request processing pipeline of FIG. 4 may be implemented, for example, as request processing pipeline 108 of cognitive processing system 100 in FIG. 1. It should be appreciated that the stages of the request processing pipeline shown in FIG. 4 are implemented as one or more software engines, components, or the like, which are configured with logic for implementing the functionality attributed to the particular stage. Each stage is implemented using one or more of such software engines, components or the like. The software engines, components, etc. are executed on one or more processors of one or more data processing systems or devices and utilize or operate on data stored in one or more data storage devices, memories, or the like, on one or more of the data processing systems. The request processing pipeline of FIG. 4 is augmented, for example, in one or more of the stages to implement the improved mechanism of the illustrative embodiments described hereafter, additional stages may be provided to implement the improved mechanism, or separate logic from the pipeline 400 may be provided for interfacing with the pipeline 400 and implementing the improved functionality and operations of the illustrative embodiments.

In the depicted example, request processing pipeline 400 is implemented in a Question Answering (QA) system. The description that follows refers to the cognitive system pipeline or request processing pipeline as a QA system; however, aspects of the illustrative embodiments may be applied to other request processing systems, such as Web search engines that return semantic passages from a corpus of documents.

As shown in FIG. 4, the request processing pipeline 400 comprises a plurality of stages 410-490 through which the cognitive system operates to analyze an input question and generate a final response. In an initial question input stage, the QA system receives an input question 410 that is presented in a natural language format. That is, a user inputs, via a user interface, an input question for which the user wishes to obtain an answer, e.g., “What medical treatments for diabetes are applicable to a 60 year old patient with cardiac disease?” In response to receiving the input question 410, the next stage of the QA system pipeline 400, i.e., the question and topic analysis stage 420, analyzes the input question using natural language processing (NLP) techniques to extract major elements from the input question, and classify the major elements according to types, e.g., names, dates, or any of a plethora of other defined topics. For example, in the example question above, “medical treatments” may be associated with pharmaceuticals, medical procedures, holistic treatments, or the like, “diabetes” identifies a particular medical condition, “60 years old” indicates an age of the patient, and “cardiac disease” indicates an existing medical condition of the patient.

In addition, the extracted major features include key words and phrases classified into question characteristics, such as the focus of the question, the lexical answer type (LAT) of the question, and the like. As referred to herein, a lexical answer type (LAT) is a word in, or a word inferred from, the input question that indicates the type of the answer, independent of assigning semantics to that word. For example, in the question “What maneuver was invented in the 1500s to speed up the game and involves two pieces of the same color?,” the LAT is the string “maneuver.” The focus of a question is the part of the question that, if replaced by the answer, makes the question a standalone statement. For example, in the question “What drug has been shown to relieve the symptoms of attention deficit disorder with relatively few side effects?,” the focus is “What drug” since if this phrase were replaced with the answer it would generate a true sentence, e.g., the answer “Adderall” can be used to replace the phrase “What drug” to generate the sentence “Adderall has been shown to relieve the symptoms of attention deficit disorder with relatively few side effects.” The focus often, but not always, contains the LAT. On the other hand, in many cases it is not possible to infer a meaningful LAT from the focus.

Referring again to FIG. 4, the identified major elements of the question are then used during a hypothesis generation stage 440 to decompose the question into one or more search queries that are applied to the corpora of data/information 445 in order to generate one or more hypotheses. The queries are applied to one or more text indexes storing information about the electronic texts, documents, articles, websites, and the like, that make up the corpus of data/information, e.g., the corpus of data 106 in FIG. 1. The queries are applied to the corpus of data/information at the hypothesis generation stage 440 to generate results identifying potential hypotheses for answering the input question, which can then be evaluated. That is, the application of the queries results in the extraction of portions of the corpus of data/information matching the criteria of the particular query. These portions of the corpus are then analyzed and used in the hypothesis generation stage 440, to generate hypotheses for answering the input question 410. These hypotheses are also referred to herein as “candidate answers” for the input question. For any input question, at this stage 440, there may be hundreds of hypotheses or candidate answers generated that may need to be evaluated.

The QA system pipeline 400, in stage 450, then performs a deep analysis and comparison of the language of the input question and the language of each hypothesis or “candidate answer,” as well as performs evidence scoring to evaluate the likelihood that the particular hypothesis is a correct answer for the input question. This involves evidence retrieval 451, which retrieves passages from corpora 445. Hypothesis and evidence scoring phase 450 uses a plurality of scoring algorithms, each performing a separate type of analysis of the language of the input question and/or content of the corpus that provides evidence in support of, or not in support of, the hypothesis. Each scoring algorithm generates a score based on the analysis it performs which indicates a measure of relevance of the individual portions of the corpus of data/information extracted by application of the queries as well as a measure of the correctness of the corresponding hypothesis, i.e. a measure of confidence in the hypothesis. There are various ways of generating such scores depending upon the particular analysis being performed. In general, however, these algorithms look for particular terms, phrases, or patterns of text that are indicative of terms, phrases, or patterns of interest and determine a degree of matching with higher degrees of matching being given relatively higher scores than lower degrees of matching.

For example, an algorithm may be configured to look for the exact term from an input question or synonyms to that term in the input question, e.g., the exact term or synonyms for the term “movie,” and generate a score based on a frequency of use of these exact terms or synonyms. In such a case, exact matches will be given the highest scores, while synonyms may be given lower scores based on a relative ranking of the synonyms as may be specified by a subject matter expert (person with knowledge of the particular domain and terminology used) or automatically determined from frequency of use of the synonym in the corpus corresponding to the domain. Thus, for example, an exact match of the term “movie” in content of the corpus (also referred to as evidence, or evidence passages) is given a highest score. A synonym of movie, such as “motion picture” may be given a lower score but still higher than a synonym of the type “film” or “moving picture show.” Instances of the exact matches and synonyms for each evidence passage may be compiled and used in a quantitative function to generate a score for the degree of matching of the evidence passage to the input question.

Thus, for example, a hypothesis or candidate answer to the input question of “What was the first movie?” is “The Horse in Motion.” If the evidence passage contains the statements “The first motion picture ever made was ‘The Horse in Motion’ in 1878 by Eadweard Muybridge. It was a movie of a horse running,” and the algorithm is looking for exact matches or synonyms to the focus of the input question, i.e. “movie,” then an exact match of “movie” is found in the second sentence of the evidence passage and a highly scored synonym to “movie,” i.e. “motion picture,” is found in the first sentence of the evidence passage. This may be combined with further analysis of the evidence passage to identify that the text of the candidate answer is present in the evidence passage as well, i.e. “The Horse in Motion.” These factors may be combined to give this evidence passage a relatively high score as supporting evidence for the candidate answer “The Horse in Motion” being a correct answer.

It should be appreciated that this is just one simple example of how scoring can be performed. Many other algorithms of various complexities may be used to generate scores for candidate answers and evidence without departing from the spirit and scope of the present invention.

In answer ranking stage 460, the scores generated by the various scoring algorithms are synthesized into confidence scores or confidence measures for the various hypotheses. This process involves applying weights to the various scores, where the weights have been determined through training of the statistical model employed by the QA system and/or dynamically updated. For example, the weights for scores generated by algorithms that identify exactly matching terms and synonyms may be set relatively higher than other algorithms that evaluate publication dates for evidence passages.

The weighted scores are processed in accordance with a statistical model generated through training of the QA system that identifies a manner by which these scores may be combined to generate a confidence score or measure for the individual hypotheses or candidate answers. This confidence score or measure summarizes the level of confidence that the QA system has about the evidence that the candidate answer is inferred by the input question, i.e. that the candidate answer is the correct answer for the input question.

The resulting confidence scores or measures are processed by answer ranking stage 460, which compares the confidence scores and measures to each other, compares them against predetermined thresholds, or performs any other analysis on the confidence scores to determine which hypotheses/candidate answers are the most likely to be the correct answer to the input question. The hypotheses/candidate answers are ranked according to these comparisons to generate a ranked listing of hypotheses/candidate answers (hereafter simply referred to as “candidate answers”).

Supporting evidence collection phase 470 collects evidence that supports the candidate answers from answer ranking phase 460. From the ranked listing of candidate answers in stage 460 and supporting evidence from supporting evidence collection stage 470, NL system pipeline 400 generates a final answer, confidence score, and evidence 490, or final set of candidate answers with confidence scores and supporting evidence, and outputs answer, confidence, and evidence 490 to the submitter of the original input question 410 via a graphical user interface or other mechanism for outputting information.

In accordance with the illustrative embodiment, medical condition extraction component 462 receives a patient electronic medical record 461, as well as other data sources containing information about the patient (not shown). These other data sources may include, for example, social networking interactions, electronic mail communications, instant messaging communications, and the like. Medical condition extraction component 462 performs natural language processing and feature extraction on patient EMR 461 and the other sources of patient data to extract features about the patient. Medical condition extraction component 462 determines medical conditions associated with the particular patient based on the extracted patient features using medical extraction model 465, which is a machine learning model that is trained to identify medical conditions based on patient features.

Content indicator association component 463 correlates medical conditions associated with the user to medical conditions for which data structures have been defined that specify the particular terms/phrases, metadata, or other indicators of content that are indicative of content of particular interest to users having the identified medical conditions. That is, content indicator association component 463 correlates the patient's medical conditions with content indicators in the answers generated by hypothesis and evidence scoring phase 450 and supporting evidence gathered by supporting evidence collection phase 470. In one embodiment, content indicator association component 463 correlates medical conditions to content indicators using user specific dictionary data structure 466, which specifies the particular content indicators associated with the patient.

Medical condition ranking component 464 then modifies scoring or ranking of candidate answers generated that were ranked by answering ranking stage 460. In one embodiment, medical condition ranking component 464 modifies scoring of the candidate answers based on the degree of matching or number of instances of matching between the content indicators associated with the patient's medical conditions, such as by modifying weights of features of the supporting evidence. In another embodiment, medical condition ranking component 464 re-ranks the candidate answers based on the degree of matching or number of instances of matching between the content indicators associated with the patient's medical conditions, increasing the scores of answers matching the content indicators and decreasing the scores of answers that do not match the content indicators associated with the patient's medical conditions.

FIG. 5 is a block diagram of a system for training a medical condition extraction model in accordance with an illustrative embodiment. Medical condition extraction model training system 510 receives labeled training data 501, which may include patient electronic medical record (EMR) data, social network interactions, electronic mail communications, instant messaging communications, and the like. Natural language processing component 511 performs natural language processing, such as deep parsing and semantic understanding of the labeled training data 501. Feature extraction component 512 extracts features relevant to medical condition identification from the labeled training data 501. Machine learning component 513 then trains medical condition extraction model 515 based on the extracted features from feature extraction component 512 and the labels in the labeled training data 501.

Medical condition extraction model 515 may be a machine learning model, such as a neural network or linear regression model. In one embodiment, medical condition extraction model 515 is a classifier that determines whether each patient from labeled training data 501 can be classified in each category, where each category is a particular medical condition, sub-type of medical condition, particular types of symptoms, etc. Medical conditions may be any condition that affects the health of the patient, including medical problems (e.g., obesity, diabetes, heart conditions, high blood pressure, etc.), behavior conditions (e.g., negative habits, overeating, alcoholism, drug addiction, etc.), and psychological conditions (e.g., phobias, compulsions, etc.). Medical condition extraction model 515 may then be used by a cognitive computing system to extract medical conditions from patient data records, such as electronic medical records (EMRs).

FIG. 6 is a block diagram of a cognitive computing system for response ranking based on personal medical condition in accordance with an illustrative embodiment. Cognitive computing system 610 receives a user request 601 from a user. In one embodiment, the user is a patient asking a question or making a request of the cognitive system 610. In another embodiment, the user is a doctor treating a patient.

Medical condition extraction component 611 applies medical condition extraction model 621 to patient data for the patient. The patient data may be, for example, electronic medical record (EMR) data, social network interactions, electronic mail communications, instant messaging communications, and the like. Medical condition extraction component 611 performs natural language on the patient data and extracts features from the patient data. Medical condition extraction component 611 then applies medical condition extraction model 621 to the extracted features to identify the patient's medical conditions.

Content indicator association component 612 correlates the extracted medical conditions to content indicators of content, which are indicative of content of particular interest to users having the extracted medical conditions. User specific dictionary data structure 622 is pre-existing and assists in the correlation.

Response generation component 613 generates candidate responses to the user request 601 from a corpus or corpora of information, as described above with reference to FIG. 4. In one embodiment, response generation component 613 also scores and ranks the candidate responses based on confidence scores of the generated responses.

User interface generation component 614 generates user interface 625, which presents the extracted medical conditions to the user. The user interface 625 allows the user to turn on or off individual medical conditions to be considered during response ranking. Thus, user interface 625 may present a list of the extracted medical conditions, each with a selection control, such as a checkbox control. If the user checks a checkbox, then the associated medical condition will be considered in ranking the candidate responses.

Response ranking component 615 calculates confidence scores for the set of candidate answers based on how well the candidate answers and/or supporting evidence match the user request. In one embodiment, response ranking component 615 ranks the set of generated candidate answers and then re-ranks the candidate answers based on the selected medical conditions. In another embodiment, response ranking component 615 scores and ranks the set of candidate answers based at least in part on the selected medical conditions. Then, cognitive system 610 outputs the resulting ranked responses 630.

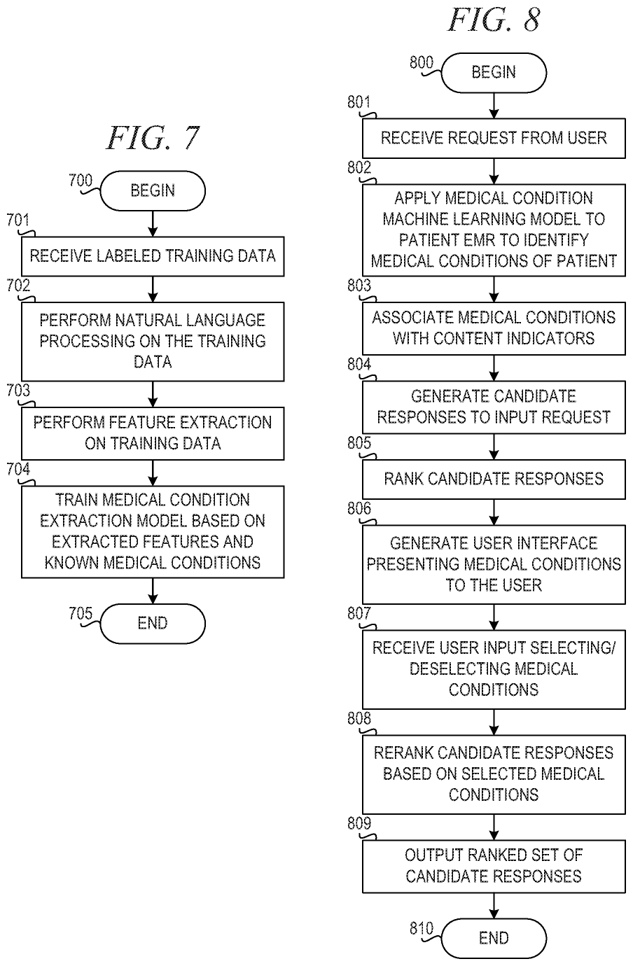

FIG. 7 is a flowchart illustrating operation of a mechanism for training a medical condition extraction model in accordance with an illustrative embodiment. Operation begins (block 700), and the mechanism receives labeled training data (block 701). The mechanism performs natural language processing on the training data (block 702) and performs feature extraction on the training data (block 703). Then, the mechanism trains a medical condition extraction model based on the extracted features and the known medical conditions of the patients in the labeled training data (block 704). Thereafter, operation ends (block 705).

FIG. 8 is a flowchart illustrating operation of a mechanism for cognitive system candidate response ranking based on personal medical condition in accordance with an illustrative embodiment. Operation begins (bock 800), and the mechanism receives a request from a user (block 801). The mechanism applies a medical condition extraction machine learning model to patient EMR and other sources of patient information to identify medical conditions of the patient (block 802). The mechanism associates the medical conditions with content indicators in the content, such as documents in a corpus of documents (block 803). The mechanism then generates candidate responses to the input request based on a corpus of content or documents (block 804) and ranks the candidate responses (block 805).

The mechanism then generates a user interface presenting the medical conditions to the user (block 806) and receives user input selecting or deselecting the medical conditions in the user interface (block 807). Then, the mechanism re-ranks the candidate responses based on the selected medical conditions (block 808) and outputs the ranked set of candidate responses (block 809). Thereafter, operation ends (block 810).

As noted above, it should be appreciated that the illustrative embodiments may take the form of an entirely hardware embodiment, an entirely software embodiment or an embodiment containing both hardware and software elements. In one example embodiment, the mechanisms of the illustrative embodiments are implemented in software or program code, which includes but is not limited to firmware, resident software, microcode, etc.

A data processing system suitable for storing and/or executing program code will include at least one processor coupled directly or indirectly to memory elements through a communication bus, such as a system bus, for example. The memory elements can include local memory employed during actual execution of the program code, bulk storage, and cache memories which provide temporary storage of at least some program code in order to reduce the number of times code must be retrieved from bulk storage during execution. The memory may be of various types including, but not limited to, ROM, PROM, EPROM, EEPROM, DRAM, SRAM, Flash memory, solid state memory, and the like.

Input/output or I/O devices (including but not limited to keyboards, displays, pointing devices, etc.) can be coupled to the system either directly or through intervening wired or wireless I/O interfaces and/or controllers, or the like. I/O devices may take many different forms other than conventional keyboards, displays, pointing devices, and the like, such as for example communication devices coupled through wired or wireless connections including, but not limited to, smart phones, tablet computers, touch screen devices, voice recognition devices, and the like. Any known or later developed I/O device is intended to be within the scope of the illustrative embodiments.

Network adapters may also be coupled to the system to enable the data processing system to become coupled to other data processing systems or remote printers or storage devices through intervening private or public networks. Modems, cable modems and Ethernet cards are just a few of the currently available types of network adapters for wired communications. Wireless communication-based network adapters may also be utilized including, but not limited to, 802.11 a/b/g/n wireless communication adapters, Bluetooth wireless adapters, and the like. Any known or later developed network adapters are intended to be within the spirit and scope of the present invention.

The description of the present invention has been presented for purposes of illustration and description and is not intended to be exhaustive or limited to the invention in the form disclosed. Many modifications and variations will be apparent to those of ordinary skill in the art without departing from the scope and spirit of the described embodiments. The embodiment was chosen and described in order to best explain the principles of the invention, the practical application, and to enable others of ordinary skill in the art to understand the invention for various embodiments with various modifications as are suited to the particular use contemplated. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, in a data processing system comprising at least one processor and at least one memory, wherein the at least one memory comprises instructions that are executed by the at least one processor to configure the at least one processor to implement a medical condition-based question answering (QA) system, the method comprising:
processing, by the medical condition-based QA system, a natural language input question about a patient to generate a set of candidate answers with an initial ranking of the candidate answers;
analyzing, by a content indicator association component of the medical condition-based QA system, portions of content associated with each of the candidate answers in the set of candidate answers based on medical condition content indicator data structures corresponding to the one or more medical conditions associated with the patient to determine which portions of content match content indicators of the medical condition content indicator data structures;
ranking, by a response ranking component of the medical condition-based QA system, candidate answers in the set of candidate answers based on the matching of content indicators of the medical condition content indicator data structures to the portions of content associated with the candidate answers to generate re-ranked candidate answers having a modified ranking; and
outputting, by the medical condition-based QA system, the re-ranked candidate answers.

2. The method of claim 1, further comprising:
analyzing, by a content indicator association component of the medical condition-based QA system, patient information associated with the patient to identify one or more medical conditions associated with the patient; and
correlating, by the content indicator association component, the one or more medical conditions with medical condition content indicator data structures, wherein each medical condition content indicator data structure comprises one or more content indicators identifying content that is of particular interest to users having a corresponding medical condition.

3. The method of claim 2, wherein analyzing the patient information comprises applying a medical condition extraction machine learning model to the patient information to identify the one or more medical conditions associated with the patient.

4. The method of claim 3, further comprising training the medical condition extraction machine learning model, comprising:
receiving a labeled training data set;
performing natural language processing on the labeled training data set;
performing feature extraction on the labeled training data set; and
training the medical condition extraction machine learning model based on the extracted features and known medical conditions in the labeled training data set.

5. The method of claim 4, wherein performing natural language processing on the labeled training data set comprises identifying recognizable medical codes.

6. The method of claim 1, wherein the one or more medical conditions associated with the patient comprise medical problems, behavior conditions, or psychological conditions.

7. The method of claim 6, wherein the one or more medical conditions associated with the patient comprise sub-types of medical conditions.

8. The method of claim 1, further comprising:
generating a user interface that presents the one or more medical conditions associated with the patient to the user; and
receiving user input selecting at least one of the one or more medical conditions associated with the patient, wherein analyzing the portions of content associated with each of the candidate answers comprises analyzing the portions of content based on medical condition content indicator data structures corresponding to the selected at least one medical condition to determine which portions of content match content indicators of the medical condition content indicator data structures.

9. The method of claim 1, further comprising generating a user specific dictionary data structure specifying content indicators for the patient based on correlation of the one or more medical conditions associated with the patient and the medical condition content indicator data structures.

10. The method of claim 1, wherein the medical condition content indicator data structures specify terms/phrases, metadata, or other indicators of content that are indicative of content of particular interest to patients having the corresponding medical conditions.

11. A computer program product comprising a computer readable storage medium having a computer readable program stored therein, wherein the computer readable program, when executed on a computing device, causes the computing device to implement a medical condition-based question answering (QA) system, wherein the computer readable program causes the computing device to:
process, by the medical condition-based QA system, a natural language input question about a patient to generate a set of candidate answers with an initial ranking of the candidate answers;
analyze, by a content indicator association component of the medical condition-based QA system, portions of content associated with each of the candidate answers in the set of candidate answers based on medical condition content indicator data structures corresponding to the one or more medical conditions associated with the patient to determine which portions of content match content indicators of the medical condition content indicator data structures;
rank, by a response ranking component of the medical condition-based QA system, candidate answers in the set of candidate answers based on the matching of content indicators of the medical condition content indicator data structures to the portions of content associated with the candidate answers to generate re-ranked candidate answers having a modified ranking; and
output, by the medical condition-based QA system, the re-ranked candidate answers.

12. The computer program product of claim 11, wherein the computer readable program further causes the computing device to:
analyze, by a content indicator association component of the medical condition-based QA system, patient information associated with the patient to identify one or more medical conditions associated with the patient; and
correlate, by the content indicator association component, the one or more medical conditions with medical condition content indicator data structures, wherein each medical condition content indicator data structure comprises one or more content indicators identifying content that is of particular interest to users having a corresponding medical condition.

13. The computer program product of claim 12, wherein analyzing the patient information comprises applying a medical condition extraction machine learning model to the patient information to identify the one or more medical conditions associated with the patient.

14. The computer program product of claim 13, wherein the computer readable program further causes the computing device to train the medical condition extraction machine learning model, comprising:
receiving a labeled training data set;
performing natural language processing on the labeled training data set;
performing feature extraction on the labeled training data set; and
training the medical condition extraction machine learning model based on the extracted features and known medical conditions in the labeled training data set.

15. The computer program product of claim 14, wherein performing natural language processing on the labeled training data set comprises identifying recognizable medical codes.

16. The computer program product of claim 11, wherein the one or more medical conditions associated with the patient comprise medical problems, behavior conditions, or psychological conditions.

17. The computer program product of claim 16, wherein the one or more medical conditions associated with the patient comprise sub-types of medical conditions.

18. The computer program product of claim 11, wherein the computer readable program further causes the computing device to:
generate a user interface that presents the one or more medical conditions associated with the patient to the user, and
receive user input selecting at least one of the one or more medical conditions associated with the patient, wherein analyzing the portions of content associated with each of the candidate answers comprises analyzing the portions of content based on medical condition content indicator data structures corresponding to the selected at least one medical condition to determine which portions of content match content indicators of the medical condition content indicator data structures.

19. The computer program product of claim 11, wherein the computer readable program further causes the computing device to generate a user specific dictionary data structure specifying content indicators for the patient based on correlation of the one or more medical conditions associated with the patient and the medical condition content indicator data structures.

20. An apparatus comprising:
at least one processor; and
a memory coupled to the at least one processor, wherein the memory comprises instructions, which when executed by the at least one processor cause the at least one processor to implement a medical condition-based question answering (QA) system, wherein the instructions cause the at least one processor to:
process, by the medical condition-based QA system, a natural language input question about a patient to generate a set of candidate answers with an initial ranking of the candidate answers;
analyze, by a content indicator association component of the medical condition-based QA system, portions of content associated with each of the candidate answers in the set of candidate answers based on medical condition content indicator data structures corresponding to the one or more medical conditions associated with the patient to determine which portions of content match content indicators of the medical condition content indicator data structures;
rank, by a response ranking component of the medical condition-based QA system, candidate answers in the set of candidate answers based on the matching of content indicators of the medical condition content indicator data structures to the portions of content associated with the candidate answers to generate re-ranked candidate answers having a modified ranking; and
output, by the medical condition-based QA system, the re-ranked candidate answers.
